(19) United States
(12) Patent Application Publication
(10) Pub. No.: US 2010/0204686 A1
(43) Pub. Date: Aug. 12, 2010

(54) LIGHT TREATMENTS FOR ACNE AND OTHER DISORDERS OF FOLLICLES

(75) Inventors: Ilya Yaroslavksy, North Andover, MA (US); Gregory B. Altshuler, Lincoln, MA (US); Valery V. Tuchin, Saratov (RU)

Correspondence Address: PALOMAR MEDICAL TECHNOLOGIES;NUTTER, MCCLENNEN & FISH LLP, SEAPORT WEST, 155 SEAPORT BOULEVARD, BOSTON, MA, 02210, US

(73) Assignee: PALOMAR MEDICAL TECHNOLOGIES, INC., Burlington, MA (US)

(21) Appl. No.: 12/724,921

(22) Filed: Mar. 16, 2010

Related U.S. Application Data
(63) Continuation of application No. 10/740,907, filed on Dec. 19, 2003, now abandoned.
(60) Provisional application No. 60/435,340, filed on Dec. 20, 2002.

(51) Int. Cl. A61B 18/18

(52) U.S. Cl. 606/9

(57) ABSTRACT

The present invention provide methods for treating acne by exposing affected follicles to at least one, and preferably two or all three, of the following radiation pulses: a radiation pulse (PC pulse) having a wavelength components in a range of about 360-700 nm; a radiation pulse (PTV pulse) having wavelength components in a range of about 470 nm to 650 nm and/or in a range of about 500 nm to about 620 nm; and a radiation pulse (PTIR pulse) having wavelength components in a range of about 900 nm to about 1800 nm. The irradiated treatment region is preferably maintained at a temperature of about 38 to 43 C in order to enhance the efficacy of the treatment.

### RELATED APPLICATIONS

The present application is a continuation of U.S. Patent Application entitled “Light Treatments for Acne and Other Disorders of Follicles” having Ser. No. 10/740,907, filed Dec. 19, 2003, which claims priority to provisional application entitled “Light Treatments for Acne and Other Disorders of Follicles” having Ser. No. 60/435,340 filed on Dec. 20, 2002, which are herein incorporated by reference.

### FIELD OF THE INVENTION

The invention relates generally to methods and systems for treatment of acne by utilizing visible and invisible electromagnetic radiation.

### BACKGROUND OF THE INVENTION

Acne is one of the most common dermatological conditions. Acne is associated with dysfunction of sebaceous follicles and/or hair follicles (mostly their sebaceous gland and infundibulum). Sebaceous glands are small oil-producing glands present in human skin. A sebaceous gland is usually a part of a sebaceous follicle (which is one type of follicle), which also includes (but is not limited to) a sebaceous duct and a pilary canal. A follicle may contain an atrophic hair (such a follicle being the most likely follicle in which acne occurs), a vellus hair (such a follicle being a less likely follicle in which acne may develop), or may contain a normal hair (acne does not normally occur in such follicles). Therefore, the teachings of this invention are primarily directed to, but are not limited to, treatment of follicles containing atrophic hairs.

FIG. 2 shows an illustrative atrophic follicle 20 located in a patient's skin having an epidermis 21 and a dermis 22. The follicle in this figure includes a sebaceous gland 23 having a basement membrane or epithelium walls 28 which generate sebocites 25, the gland typically being located at a depth of approximately 0.7 to 2 mm from the skin surface. The gland is connected to the follicle canal by a sebaceous duct 26 having an epithelial lining 29, and an infundibulum 31, which is the portion of the follicle canal above the duct. The lower portion of the infundibulum is referred to as the infrainfundibulum 27 in which comedones can be formed. This process is initially manifested as abnormalities in the kearatinization and desquamation of the epithelial cells (keratinized and sloughing cell 33 in the figure). The infrainfundibulum has an epithelial lining 34, an upper portion of which is the acroinfundibulum 32. An atrophic follicle may have a hair 24 as shown at the bottom of the canal.

Disorders of follicles are numerous and include acne vulgaris, which is the single most common skin affliction. Development of acne usually starts with formation of non-inflammatory acne (comedo) that occurs when the outlet from the gland to the surface of the skin is plugged, allowing sebum to accumulate in the gland, sebaceous duct, and pilary canal. Although exact pathogenesis of acne is still debated, it is firmly established that comedo formation involves a significant change in the formation and desquamation of the keratinized cell layer inside the infrainfundibulum. Specifically, the comedos can form as a result of defects in both desquamating mechanism (abnormal cell cornification) and mitotic activity (increased proliferation) of cells of the epithelial lining of the infrainfundibulum.

The chemical breakdown of triglycerides in the sebum, predominantly by bacterial action, releases free fatty acids, which in turn trigger an inflammatory reaction producing the typical lesions of acne. Among microbial population of pilosebaceous unit, most prominent is Propionibacterium Acnes (P. Acnes). These bacteria are causative in forming inflammatory acne.

A variety of medicines are available for acne. Topical or systemic antibiotics are the mainstream of treatment. Oral isotretinoin is a very effective agent used in severe cases. However, an increasing antibiotic resistance of P. Acnes has been reported by several researchers, and significant side effects of isotretinoin limit its use. As a result, the search continues for efficient acne treatments with at most minimal side effects, and preferably with no side effects. To this end, several techniques utilizing light have been proposed. For example, one such method utilizes laser sensitive dyes for treatment of sebaceous gland disorders. More particularly, this method calls for applying a chromophore-containing composition to a section of the skin surface, letting a sufficient amount of the composition penetrate into spaces in the skin, and exposing the skin section to (light) energy causing the composition to become photochemically or photothermally activated. A similar technique involves exposing the subject afflicted with acne to ultraviolet light having a wavelength between 320 and 350 mu.

The use of blue (wavelength 415 nm) and red (660 nm) light for phototherapy of acne has also been reported. A method of treating acne with at least one light-emitting diode operating at continuous-wave (CW) mode and at a wavelength of 660 nm is also known. This treatment represents a variation of photodynamic therapy (PDT) with an endogenous photosensitizing agent. Specifically, P. Acnes are known to produce porphyrins (predominantly, coproporphyrin), which are effective photosensitizers. When irradiated by light with a wavelength strongly absorbed by the photo sensitizer, this molecule can give rise to a process known as the generation of singlet oxygen. The singlet oxygen acts as an aggressive oxidant on surrounding molecules. This process eventually leads to destruction of bacteria and clinical improvement of the condition.

Another method of reducing sebum production in human skin utilizes pulsed light with a range of wavelengths that is substantially absorbed by the lipid component of the sebum. The postulated mechanism of action is photothermolysis of differentiated and mature sebocytes.

The existing light-employing treatment techniques, however, suffer from at least the following drawbacks:
1. Blue light (400 to 450 nm), most effectively absorbed by porphyrins (See FIG. 1), has very limited penetration depth in normal blood-containing skin. More precisely, the penetration depth of such light does not exceed −300 gm, whereas the population density of P. Acnes (primary target of the PDT) peaks at -1.2 mm depth.
2. Thermal effect can cause difficulty in achieving maximal efficacy of the PDT treatment. Specifically, mild hyperthermia has been shown to increase the efficacy of PDT. However, a rise of temperature above −43° C. initiates the process of tissue coagulation and decreases the efficacy of treatment. In addition, overheating of tissue makes the process painful. Therefore, there exists an upper limit on the irradiance that can be delivered to the target under normal conditions (−200 mW/cm² for CW or quasi-CW treatment), thereby limiting the rate of singlet oxygen generation. At the same time, the irradiance levels typically provided by LED(s) (between 10 and 30 mW/cm²) are sub-optimal for achieving maximal efficiency of singlet oxygen generation and require unreasonably long treatment times.
3. Photothermal treatment with light of wavelengths predominantly absorbed by the lipid component of sebum leads only to photocoagulation of sebocytes and their elements and does not reduce hyperproliferation and abnormal comification in the epithelial lining of the infrainfundibulum and sebaceous duct. As a result, such a treatment does not necessarily reduce the probability of comedo formation and can even be counter-productive by solidifying the plug.
4. PDT alone does not eliminate the original cause of acne, i.e. plugging of the sebum outlet and accumulation of excessive sebum in the sebaceous duct. At the same time, the thermal treatment alone does not necessarily reduce the bacteria population, which can lead to relapse of the disease.

Hence, there is a need for enhanced methods for treating acne.

There is also a need for systems that can readily implement such methods.

### SUMMARY OF THE INVENTION

In one aspect, the present invention provides a method for treating a follicle that includes irradiating a portion of a patient's skin surface by one or more pulses of electromagnetic radiation so as to expose a treatment region of the follicle to the radiation. The radiation is selected to have one or more wavelength components suitable for causing selected photochemical effects on bacteria and/or cells in the treatment region so as to treat a skin condition, such as acne. During application of the treatment radiation, the temperature of the treatment region is maintained in a range of about 38 C to about 43 C so as to enhance the efficacy of the applied radiation.

In a related aspect, the applied radiation pulse(s), herein also referred to as a photochemical (PC) pulse, has wavelength components in a range of about 380 nm to about 700 nm. More preferably, the radiation pulse has wavelength components in at least one of the following ranges: about 380 nm to about 430 nm; about 480 nm to about 510 nm; and about 600 nm to about 700 nm. The radiation pulse can also include wavelength components, e.g., wavelengths components in a range of about 900 nm to about 1400 nm, suitable for heating the treatment region so as to maintain its temperature within the above range.

In another aspect, the pulse duration is selected to be in a range of about 1 ms to about 20000 ms, or more preferably, in a range of about 20 ms to about 1000 ms. Further, the pulse can provide a radiant exposure in a range of about 2 to about 50 J/cm², or more preferably, in a range of about 2 to about 20 J/cm².

In another aspect, the treatment region includes any of a sebaceous gland, a sebaceous duct and/or the infrainfundibulum of the follicle. Multiple pulses, each having wavelength components within one of the above ranges, can also be utilized to treat the follicle. Such pulses can be applied to the treatment region simultaneously or sequentially.

In another aspect, pressure, for example, in a range of about 10 to about 100 Newton/cm², is applied to the irradiated skin portion during treatment so as to reduce tissue inhomogeneity, expel blood from skin vessels, and/or reduce travel distance of the radiation to the treatment region, thereby increasing penetration depth of the applied radiation.

In another aspect, the invention provides a method for treating a follicle by irradiating the follicle with a pulse of electromagnetic radiation having a wavelength spectrum, a duration and a radiant energy that are selected to raise a temperature of at least some epithelial cells of the follicle to a value in a range of about 43 C to about 47 C. The method also calls for cooling at least a portion of the patient's skin through which the pulse propagates to the follicle.

In a related aspect, the wavelength spectrum of such a pulse, herein also referred to as photothermal-infrared (PTIR) pulse, spans a range of about 900 nm to about 1800 run, and more preferably, about 1000 nm to about 1600 nm. Further, the pulse can have a duration in a range of about 1 ms to about 100 seconds, and can provide a radiant exposure in a range of about 10 to about 500 J/cm².

In another aspect, the invention provide a method of treating a follicle that calls for irradiating one or more blood vessels supplying blood to the follicle with at least one pulse of electromagnetic radiation having a wavelength spectrum, a duration, and a radiant energy selected so as to reduce generation of sebum in the follicle's gland, and cooling at least a portion of skin surface through which said pulse propagates to irradiate said vessels.

In a related aspect, the pulse, herein referred to also as a photothermal-visible (PTV) pulse, can have wavelength components in a range of about 470 nm to 650 nm, and more preferably, in a range of about 500 nm to about 620 nm. The PTV pulse can have a duration in a range of about 0.1 ms to 1000 ms, and more preferably, in a range of about 1 ms to 100 ms, and can provide a total radiant exposure in a range of about 10 to about 50 J/cm².

In other aspects, the invention provides dermatological systems for implementing the above methods of the invention. A dermatological system, as used herein, can refer to a therapeutic system or a cosmetic system, including a home cosmetic system. One such system according to the teachings of the invention can include a radiation generating source for irradiating a portion of skin with at least one pulse of photochemical electromagnetic radiation so as to expose a treatment region of at least one follicle to said radiation, and a source for generating photothermal radiation to heat at least a portion of the treatment region. A source that generates a pulse of radiation can inherently function in a pulsed mode. Alternatively, it can inherently generate continuous radiation from which one or more pulses can be formed by switching of the device, e.g., via switching electronics.

In another aspect, the invention provides a handheld dermatological system for treating follicles that includes a housing with a handle and an enclosure, and at least one radiation generating source within the enclosure for irradiating a portion of skin with at least one pulse of photochemical electromagnetic radiation so as to expose a treatment region of at least one follicle to said radiation and at least one source for generating photothermal radiation also within the enclosure to heat at least a portion of the treatment region.

Further understanding of the invention can be obtained by reference to the 15 following detailed description and the associated drawings, described briefly below.

### BRIEF DESCRIPTION OF DRAWINGS

FIG. 1A is a graph illustrating a typical absorption spectrum of porphyrins.

FIG. 1B is a graph illustrating absorption spectra of major skin chromophores.

FIG. 2 is a schematic representation of a follicle and a treatment technique in accordance with the teachings of the invention.

FIG. 3 is a graph illustrating computed action spectra associated with photochemical mechanism of action for two exemplary light sources (i.e., a metal halide lamp and a Xe flashlamp), assuming a target depth of 1.2 micrometers.

FIG. 4 is a chart illustrating an exemplary dependence of irradiance and pulsewidth on the patient's skin type for a photochemical (PC) pulse.

FIG. 5 is a chart illustrating an exemplary dependence of irradiance and pulsewidth on the patient's skin type for a photothermal-visible (PTV) pulse.

FIG. 6 is a graph illustrating the distribution of the absorbed optical energy in 5 the A-A cross-section of FIG. 2 for a photothermal-infrared (PTIR) pulse.

FIG. 7 is a schematic diagram of an apparatus according to one embodiment of the invention for targeting small skin areas for acne treatment,

FIG. 8A is a schematic diagram of an apparatus according to another embodiment of the invention for targeting large skin areas (e.g., a whole face) for acne treatment,

FIG. 8B is a schematic representation of a matrix array of light sources utilized 15 in the apparatus of FIG. 8A.

FIG. 9A is a schematic diagram of an acne treatment apparatus according to the teachings of the invention in which two pulses having different spectral characteristics are simultaneously obtained by selective filtering of light generated by a single 20 broadband source,

FIG. 9B is a schematic diagram of another acne treatment apparatus according to the teachings of the invention in which two spectrally different pulses are obtained sequentially from the light generated by a single broadband source.

FIG. 9C is a schematic diagram of an acne treatment apparatus according to another embodiment of the invention in which two spectrally different pulses are obtained by utilizing light generated by two broadband sources either simultaneously or sequentially.

FIG. 9D is a schematic diagram of yet another acne treatment apparatus according to the teachings of the invention in which two spectrally different pulses are obtained from a combination of a broadband source and a laser source pumped by the broadband source.

### DETAILED DESCRIPTION OF THE INVENTION

This invention discloses how light energy can be efficiently used to treat the disorders of follicles described above. A combination of photothermal and photochemical mechanisms are used to achieve this goal by use of at least one of three treatments, one of which is optimized for photodynamic effect, whereas the other two are optimized for controlled heating of target tissue. One or more of the following steps are utilized:
1. Applying pressure to the skin surface in order to reduce tissue inhomogeneity, decrease the distance that light has to travel from the skin surface to the gland, and expel blood from the vessels in the skin (most importantly, remove blood from the dermal plexus, which normally absorbs up to 30% of incident light energy in the blue spectral region);
2. Cooling of the skin surface in order to reduce the temperature of the epidermis (thus protecting it from thermal injury) and to minimize the blood flow through the dermal vessels due to vasoconstriction;
3. Precise thermal management in order to increase the efficacy of the photodynamic process;
4. Application of an oxygen-rich or otherwise absorption-enhancing topical composition to the selected treatment area prior to treatment in order to increase the efficacy of the photodynamic process;
5. Optimization of the pulse parameters in order to achieve selective heating and, in some embodiments, photothermolysis of tissue around the sebaceous gland, sebaceous duct, and/or infrainfundibulum.

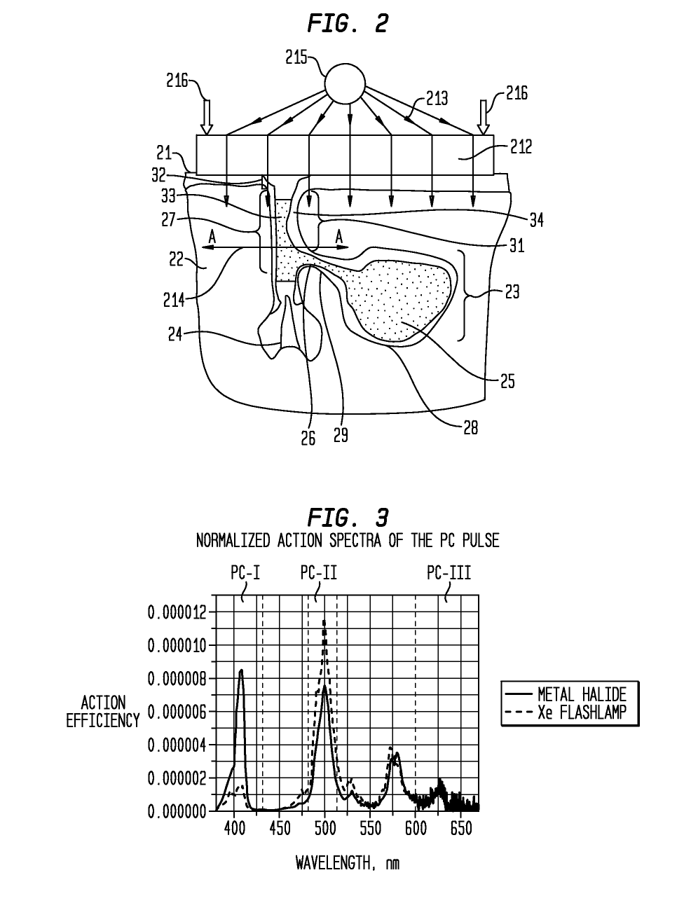

The light treatment in accordance with the teachings of this invention can include treatments with at least one, preferably two, and most preferably, three substantially different light pulses. Such a one, two, or three pulse treatments can be applied to a treatment area sequentially or coincidentally, and also cyclically. The pulses are different in their spectral composition, energy, and (in some embodiments) duration. Schematics of the follicle and a possible technique of light delivery are illustrated by FIG. 2. In this figure, an exemplary contact mechanism 212 may be used for beam shaping, cooling, as a pressure apparatus and/or to perform other functions known in the art. This contact mechanism may be, for example, a plate which is optically transparent at least at the wavelengths utilized and, where used for cooling, has good thermal properties; or may be a waveguide having similar properties. 215 is a suitable optical radiation source, suitable radiation sources being discussed hereinafter, and 213 is the radiation from the source. Optionally, pressure providing elements 216 can be utilized to press the contact mechanism against the skin in a controlled manner. Other components for control, cooling where employed, etc. would also be provided as required. While a contact mechanism is shown for a preferred embodiment, and a contact mechanism is clearly preferred, particularly where pressure is to be applied, this is not a limitation on the invention, and an embodiment using a non-contact mechanism or mechanisms to perform functions such beam shaping and cooling may also be possible.

Specifically, the three light pulses that can be utilized in a method of the invention for treating acne are:

### Photochemical (PC) Pulse

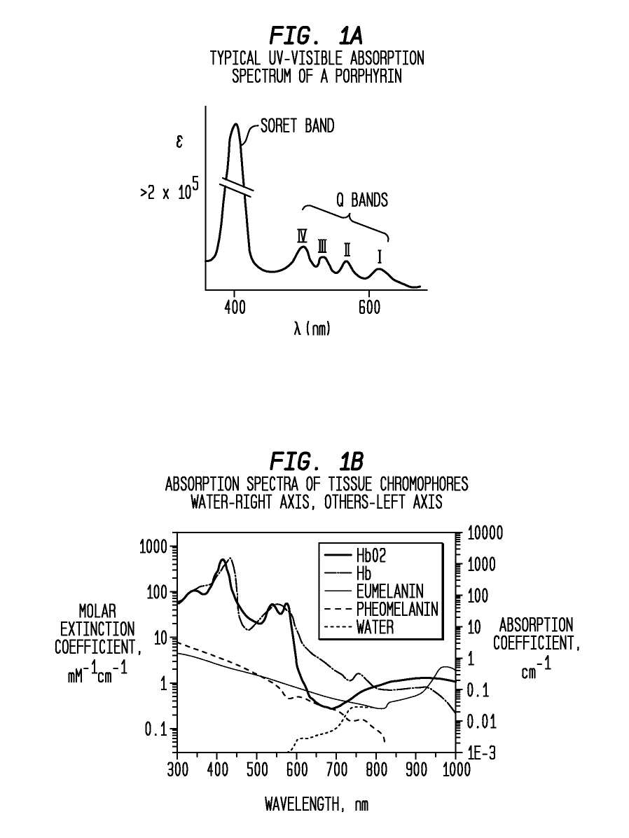

A first of the pulses, referred to as the “photochemical” (PC) pulse, is optimized to match the absorption spectrum of the target porphyrin or other photosensitizers (See FIG. 1A). Specifically, part of the original broad spectrum emitted by a broad spectrum source, such as a lamp, is filtered out in such a way as to eliminate unwanted portions of optical energy, for example energy between at least some absorption bands of the porphyrin and energy at wavelengths primarily absorbed by skin above the sebaceous gland(s) being treated, for example the epidermis, the latter causing potential thermal damage to the patient's skin while not contributing significantly to the treatment. FIG. 1B illustrates absorption spectra of major skin chromophores. For one embodiment, the PC pulse contains light in a wavelength range between 380 nm and 700 nm.

For a more preferred embodiment, the PC pulse contains light in at least one of the following wavelength ranges:
between 380 nm and 430 rim (PC-I);
between 480 nm and 510 nm (PC-II);
between 600 nm and 700 nm (PC-III)

The spectral intervals between 430 nm and 480 nm, as well as between 510 nm and 600 rim, are filtered out in this embodiment in order to better match the incident spectrum to the absorption spectrum of the target and to reduce unwanted thermal load on the epidermis and upper layers of the dermis. The upper limit of the PC pulse wavelength range (700 nm) is determined from the observation that wavelengths in the range 700-900 nm have a strong heating effect on the epidermis as a result of melanin absorption but are not significantly absorbed by porphyrins. The PC-I, PC-II, and PC-III wavelength ranges are illustrated in FIG. 3.

According to one aspect of the method of the present invention, the temperature of the target site should be maintained within a range that is optimal for PDT (between about 38° C. and about 43° C.). Therefore, in the preferred embodiment, the PC pulse also contains a portion of deep-penetrating light, preferably at a wavelength range between 900 nm and 1800 nm. Energy of this portion of the PC pulse, which is absorbed by water and lipid in tissue, is dissipated as heat and creates the desired mild hyperthermia at the target site. Therefore, in the most preferred embodiment, the PC pulse contains light in each of the following wavelength ranges:
between 380 nm and 430 nm (PC-I), predominant effect on superficial part of follicle;
between 480 nm and 510 nm (PC-II), predominant effect on intermediate part of follicle;
between 600 nm and 700 rim (PC-III), penetration to deeper part of follicle; between 900 nm and 1800 nm(preferably 900-1400 nm) (PC-H), hyperthennia at target area.

For some, but not all, skin types, the light energy may be more or less equally distributed between the three above wavelength bands; however, the energy in each wavelength band will ultimately be determined by the desired therapeutic effect, for example, the energy required to raise the treatment region to the desired temperature range for the PC effect and to maintain the region in this temperature range. In an alternative embodiment, the PC pulse can be delivered as a sequence of two sub-pulses (PC-A and PC-B), the former comprising wavelength ranges PC-I, PC-II and PC-H, and the latter comprising wavelength ranges PC-III and PC-H. While for some embodiments, the PC-B pulse may have greater energy to compensate for the lower absorption of porphyrins at these wavelengths, other factors may also be involved so that this is not always the case.

Since the spectral composition of the PC pulses should include a substantial portion of light in the visible range where tissue attenuation is strong (See FIGS. 1A and 1B), it is desirable to maximize penetration depth of this light during treatment. To this end, at least the following two techniques can be used:
application of pressure to the skin surface in order to reduce tissue inhomogeneity, to decrease the distance that light has to travel from skin surface to the gland, and to expel blood from the skin vessels; and
cooling of the skin surface in order to reduce the temperature of the epidermis and the blood flow through the superficial blood vessels in the skin.

In order to maintain the advantageous temperature regimen specified above, the method of the present invention also employs cooling of the skin surface to manage a precise temperature regimen at the depth of PDT action (−1.2 mm). Specifically, cooling parameters are adjusted in such a way as to create mild hyperthermia (preferably, between 38° C. and 43° C.) at the specified depth. The combination of an energetic PC pulse and cooling maximizes the efficacy of the PDT process. At the same time, cooling of the surface protects the epidermis from overheating and thermal damage.

The preferred duration of the PC pulse is between 1 ms and 20000 ms, more preferably 20-1000 ms with total radiant exposure between 2 and 50 J/cm2, preferably between 2 and 20 J/cm2. The pulse duration and radiant exposure are selected to maintain follicles and/or the sebaceous glands being treated within a temperature range where photochemical effects are optimized, a temperature range generally between about 38° C. and about 43° C., and to provide sufficient energy to the porphyrin to achieve maximal efficiency of the photodynamic process. Radiant exposure and duration will vary as a function of the energy spectrum of the light source being utilized, of the porphyrin being treated, of the skin characteristics of the patient and of other factors. The specific radiant exposure and duration may be determined empirically for a given treatment, the following relationship being useful in determining these parameters in the more preferred embodiment:

Radiant exposure [J/cm²]=2+(6−S)*3. 6, (Eq.1)

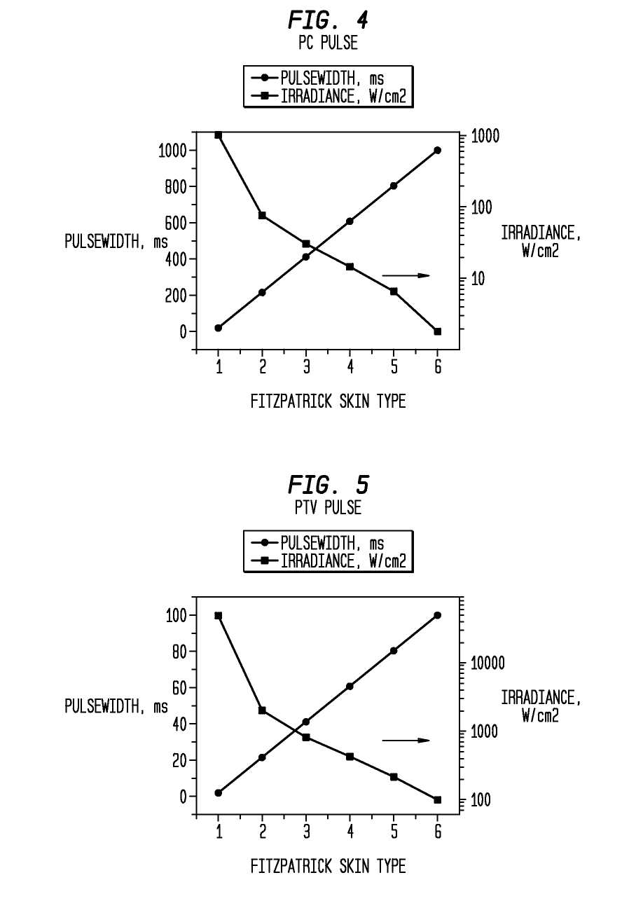

Duration [ms]=20+(S−1)*196, (Eq.2)
where S is patient's skin type according to Fitzpatrick's scale (between 1 and 6). Eqs. (1) and (2) are illustrated in FIG. 4. Eqs. (1) and (2) assume a substantially equal division of radiant exposure in the three wavelength ranges. It is understood that Egs.(1) and (2) should not be considered as limiting the scope of the present invention, and that the radiant exposure and duration given by Eqs. (1) and (2) can be adjusted for a particular treatment.

While in the discussion above, broadband radiation sources have been indicated as the radiation sources, a variety of radiation sources (including, for example, arrays of lasers, such as diode lasers, vertical cavity surface emitting lasers (VCSELs), fiber lasers; or LEDs) can be used to produce a pulse (or pulses) with the required characteristics. However, while one or more monochromatic or limited wavelength light sources can be used to generate the PC pulse(s), a broadband pulsed lamp (arc discharge, halogen, metal halide, incandescent or other) is preferably used. More preferably, a Xe pulsed flash-lamp is used as the light source for the PC pulse, with color temperature in the range between 5,000 K and 10,000 K.

The efficacy of the PC pulse can be further increased in some embodiments by application (prior to treatment) of an oxygen-rich topical composition to the area selected for treatment in order to increase concentration of oxygen available for the PDT process in the target area. The oxygen-rich composition would diffuse into the skin. For example, peroxidized corn oil can be used as an active ingredient of such a composition, and the composition can be made in the form of a gel, wax, or adhesive film. The topical composition can be applied, for example, before treatment and between treatment pulses.

The total dose can be maximized by increasing the radiant exposure of every pulse and/or providing multiple pulses to the same area. This can be implemented as a stack of pulses or as several passes of contact mechanism 212 over the same treatment area. For example, the PC-A pulse can be delivered on the first pass, and PC-B pulse can be delivered on the second pass.

### Photothermal-Visible (PTV) Pulse

The photothennal visible (PTV) pulse is designed to target blood vessels supplying follicles, including the epithelium of the sebaceous gland, other portions of the sebaceous gland, the infrainfindibulum, etc. The vascular system is particularly well developed around large follicles, which are most susceptible to camedo formation. The objective is to arrest or prevent comedo formation by reducing generation of sebum in the gland and restricting proliferation of keratinized cells. The preferred wavelength range for the PTV pulse is between 470 nm and 650 nm, most preferably between 500 mu and 620 nm. The preferred duration of the PTV pulse is between 0.1 ms and 1000 ms, more preferably between 1 ms and 100 ms, with total radiant exposure between 10 and 100 J/cm², more preferably between 10 and 50 J/cm². The specific radiant exposure and duration may be determined empirically for a given treatment, the following relationship being useful in determining these parameters:

Radiant exposure [J/cm²]=10+(6−S)*8, (Eq.3)

Duration [ms]=1+(S−1)*20, (Eq.4)
where S is patient's skin type according to Fitzpatrick's scale (between 1 and 6). Eqs. (3) and (4) are illustrated by FIG. 5. It is understood that Eqs.(3) and (4) should not be considered as limiting the scope of the present invention, and that the radiant exposure and duration given by Eqs. (3) and (4) can be adjusted for a particular treatment.

The PTV pulse can be produced either by the same light source that is used for generating the PC pulse or by a different light source. For one preferred embodiment, an Xe flashlamp is used to generate both pulses. Required pulse characteristics are achieved by varying electrical parameters of the power supply and optical filtration of the emitted light.

Surface cooling can be used during the PTV pulse in order to prevent unwanted epidermal and dermal thermal damage, to create optimal conditions for controlled thermal destruction of sebaceous gland cells and to allow delivery of more energy to the treated target.

### Photothermal-Infrared (PTIR) Pulse

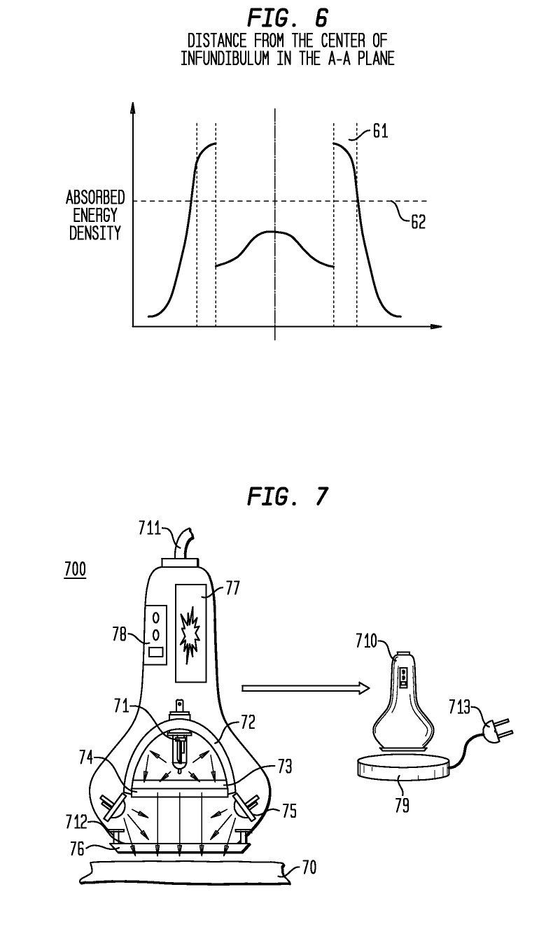

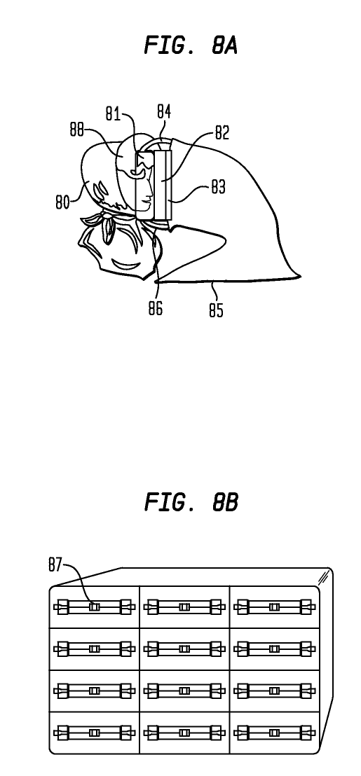

The photothermal infrared (PTIR) pulse is optimized to create controlled thermal damage within the epithelial lining of the sebaceous gland, sebaceous duct, and infrainfundibulum (28, 29, 34 respectively in FIG. 2). It targets the basal cells of the epithelium 34 in the infrainfundibulum with the aim of decreasing their mitotic activity and normalizing the cornification mechanism. The goal is achieved by creating an area of elevated temperature at the epithelium (“thermal shell”). This is possible due to differences in the optical and thermal properties of the material within the follicle (sebum and disorganized cells and cell fragments) and surrounding dermis. In particular, the ratio of the scattering coefficient to the absorption coefficient is significantly higher within the follicle, thus allowing a waveguide-like propagation of light through the pillary canal; and, further, the thermal conductivity of the material within the follicle is lower. The “thermal shell” concept is illustrated schematically by FIG. 6 (plane A-A is as illustrated in FIG. 2). In addition, it is known that epidermal and dermal cells are more resistive to elevated temperatures than the cells of the epithelium (62 in FIG. 6 being for example the energy threshold for photothermal damage in the epithelium 61), this resulting from the biological differences in structure and function of the cells. Therefore, a range of temperatures exists where these differences in biological response lead to irreversible damage to the cells of the epithelium, whereas epidermal and dermal cells remain intact. While the exact temperatures of this range will vary somewhat from patient to patient, and even for different areas of the same patient's body, depending on a number of physiological factors, this temperature range is generally about 43° C. to about 47° C. Unlike some prior art methods, the PTIR pulse of the present invention does not target the sebocites themselves. Accordingly, there is no need to select the PTIR pulse wavelengths to be predominantly absorbed by lipids. The spectral composition of the PTIR pulse should preferably meet the following requirements: Single scattering albedo of the material in the lumen of the infrainfundibulum (sebum and cell debris) at the wavelengths composing the PTIR pulse should be higher than that of the surrounding tissue. Further, absorption coefficient of the material in the lumen of the infrainfundibulum (sebum and cell debris) at the wavelengths composing the PTIR pulse should be lower than that of the surrounding tissue.
Moreover, preferably, PTIR pulse should be a substantially broadband pulse (more preferably, with spectral width >100 nm) in order to create the “thermal shell” for a sufficient range of depths, thus providing treatment for a sufficiently large number of acne-prone follicles.

Preferably, the IR pulse represents a broadband pulse within the following spectral range:
between 900 nm and 1800 nm.

More preferably, the IR pulse represents a broadband pulse within the following spectral range:
between 1000 nm and 1600 nm.

The wavelengths not filtered out are the wavelengths optimally absorbed by water, the main constituent of tissue surrounding the glands, and are thus the wavelengths which are most effective in heating the epithelium. These wavelengths also have good penetration to the depth of the sebaceous glands and are not significantly absorbed by melanin so that they cause less heating of the skin above the glands than other wavelengths.

The duration of this pulse is, for example, preferably between 1 ms and 100 s, with total radiant exposure between 10 and 500 J/cm². The duration and radiant exposure for a particular treatment are selected so as to raise the temperature of the epithelium being treated to a value generally in the range indicated above for a time interval sufficient to achieve the desired therapeutic effect. Since absorption of light in the preferred wavelength range for the PTIR pulse is almost independent of skin pigmentation, typically no adjustment of the pulse parameters to accommodate the patient's skin type is necessary. There are at least three possible desired therapeutic effects. The pulsewidth is determined depending on which of these effects is to be achieved. These three effects are (in order of increasing radiant exposure, i.e. increasing pulsewidth while maintaining a constant irradiance): reducing/eliminating sebum production from the gland, accelerating apoptosis of the epithelial cells, and destroying the epithelial cells by necrosis with subsequent replacement by scar tissue. Because of variations in sebaceous glands and other factors, one or more of these effects may occur for various glands during a given treatment.

The source of the PTIR pulse may or may not be the same as that of the PC pulse and PTV pulse. In the former case, different light sources, or preferably different filtration is used to achieve desired spectral characteristics. In a preferred embodiment, a broadband pulsed lamp (arc discharge, halogen, incandescent or other) is used. In a more preferred embodiment, a halogen lamp is used as the light source for the PTIR pulse, with color temperature in the range between 1,000 K and 4,000 K. Additional filtration of the output of the source is used in the preferred embodiment.

The order in which the pulses are delivered and the interval between treatments with PC, PTV, and PTIR pulses is not critical (e.g., the interval between the pulses can be between 100 ms and several hours). However, since the photodynamic and the photothermal treatments of the respective pulses are substantially independent, the interval may be even longer, perhaps even days, although this is not currently preferred. Multiple pulses of each type can be delivered to increase the efficacy of treatment. As with single pulses, the order of and the interval between the pulses is not critical; however, the number of pulses, their order and interval may influence duration and radiant exposure for the pulses.

Surface cooling would normally be used during the PTIR pulse in order to prevent unwanted epidermal and dermal thermal damage and create optimal conditions for controlled thermal destruction of cells of follicles and/or sebaceous glands.

Some embodiments of the invention can use application of either acoustic, radio-frequency, or microwave energy for more precise temperature management during one or more pulses. For example, such a source may be utilized during the PC pulse, either in addition to or instead of the PC-H portion of this pulse, to heat target tissue to the desired temperature range.

While the use of all three of the pulses discussed above is advantageous for treatment of certain problems of the follicles, including acne, advantageous results can also be achieved by using any one of the three pulses alone, particularly the PC pulse, or any combination of two or more of the three pulses. The use of the PC pulse enhanced by cooling, pressure and/or operating on follicle tissue in an advantageous temperature range provides significantly enhanced reduction or elimination of bacteria even if the other pulses are not employed. Similarly, the targeting of the “thermal shell” by the PTIR pulse enhances the efficacy of this pulse in reducing and/or eliminating sebum production and/or comedo formation, and thus in the treatment of acne.

A variety of different systems and apparatus can be employed for practicing the teachings of the invention. Some exemplary embodiments of apparatus suitable for implementing the methods of the invention, schematically depicted in FIGS. 7-9, are described below.

FIG. 7 schematically illustrates an apparatus 700 that can be utilized for treating a small target area 70 (e.g., up to several centimeters in diameter) that contains only a few acne lesions or even a single lesion. For example, the apparatus 700 can be employed to treat inflammatory acne for quick, e.g., overnight, reduction of inflammation. The exemplary apparatus 700 includes a light source 71 (preferably halogen or arc lamp) that is placed within a reflector 72, which directs light generated by the source 71 onto a filter 73. The filter 73 can be selected to obtain various spectral compositions of the output light, e.g., corresponding to either PC, PTV, or PTIR pulses described above. In one embodiment, the light source can be a halogen lamp, which typically has a spectrum with maximum energy between 600-2000 nm, that can be utilized for generating light for PC, PTV or PTIR treatments. A transparent element 74 provides cooling to the filter 73 and, optionally, is utilized for additional beam shaping of the filtered radiation.

The apparatus 700 further includes light emitting diodes 75 that can generate a PC and/or a PTV pulse. Further, an output window 76 can provide optical coupling to, and optionally, cooling of the irradiated skin portion. The window 76 can be cooled by an attached heat/cool capacitor (not shown) utilizing a phase change material, such as ice or wax. Alternatively, the window 76 can cooled by circulating water or by a mechanism spraying a phase change material (e.g., freon) thereon and/or on the skin. The window 76 can be spring-loaded to maintain a controlled pressure on the skin surface during the treatment protocol by utilizing pressure-inducing elements 712. In general, various cooling techniques can be employed to cool the window 76 and, possibly, the element 74. In one embodiment, the window 76 can be thermally coupled to a cooling base 79, which is powered via a cord 713 and employs thermoelectric cooling.

With continued reference to FIG. 7, the apparatus includes a power supply 77 that is located in the handle of the device, and a control panel 78 that allows an operator to control the device. In this embodiment, various components of the apparatus 700 are packaged in a hand-held ergonomic enclosure 710. The apparatus 700 is preferably cordless and employs rechargeable batteries for energy storage. For example, in this embodiment, the base 79 serves not only a cooling device but also as a charging device. Alternatively, the apparatus 700 can be powered via a power cord 71.

The apparatus 700 can be utilized by a medical professional for practicing the methods of the invention. Alternatively, a treatment regimen according to the teachings of the invention can be self-administered by employing this apparatus. For example, parameters of the light sources and the treatment regimen can be selected to achieve a rapid (e.g., within a few hours) resolution of the targeted lesions. The exemplary apparatus 700 can also be equipped with a suction mechanism (not shown) to suck inflammatory papules onto the transparent window 76 to allow better light delivery to the target. In this embodiment, the window 76 is disposable to obviate the need for sterilization between treatments.

The exemplary apparatus 700 can be utilized for home treatment. In such a case, the apparatus will be equipped with a sensor, e.g., a skin touch sensor, to prevent its activation on the eye. Such sensors can be mechanical, optical, electrical or other types of sensors. In some embodiments, the sensor is designed to activate the apparatus only when a special lotion is applied to the target skin portion. In other embodiments, the apparatus, herein also referred to as “acne pen,” can be equipped with a sensor that detects acne inflammation.

FIG. 8A schematically presents another apparatus 800 for implementing the treatment methods of the invention by targeting large areas, e.g., whole face of a patient. The exemplary apparatus 800 includes a receptacle 81 into which a patient 80 can place her face. The receptacle 81 can be, for example, an enclosure having soft, optically transparent walls. An optically transparent coolant can be circulated through the enclosure via an inlet 84 and an outlet 86. The receptacle 81 is attached to a screen 8 covering a matrix 83 of a plurality of light sources. For example, as schematically illustrated in FIG. 8B, the matrix 83 can be formed as an array of light sources, such as, flash lamps, halogen lamps, diode lasers or LEDs, which can be activated sequentially, simultaneously, or in a selected pattern. Each cell 87 of the matrix array 83 represents a light-emitting element that comprises either a single light source or multiple light sources. In some embodiments, the light from the matrix 83 can be delivered onto the treatment area through an air gap without active cooling or with active cooling of the irradiated skin portion by air flow.

Referring again to FIG. 8A, the screen 82 can be made from a material that can have spectra filtering functionality, such as a dye doped plastic. For example, the dye can be a fluorescent dye that converts the light from the matrix 83 into light having a desired spectrum. Techniques described below can be used to produce desired combinations of photochemical and photothermal pulses from each cell. The apparatus 800 further includes an enclosure 85 that can contain a power supply, control electronics, a cooling mechanism, and other auxiliary components known in the art. When sequential activation of the light sources is employed, the power supply can be made advantageously small, light-weight and inexpensive. Moreover, protective goggles can be used to eliminate the possibility of eye injury. Further, the apparatus 800 can be equipped with a mechanism for automatic eye, lips, and/or hair protection from light exposure. For example, sensors can be employed to ensure activation of the device only if the patient's eyes are protected by a shield. In addition, an acne diagnostic sensor (e.g., fluorescent sensor) can be incorporated in the device. The apparatus 800 can be utilized, for example, at home for acne treatment including acne prevention treatment and/or combined treatment of acne and skin toning an texture treatment.

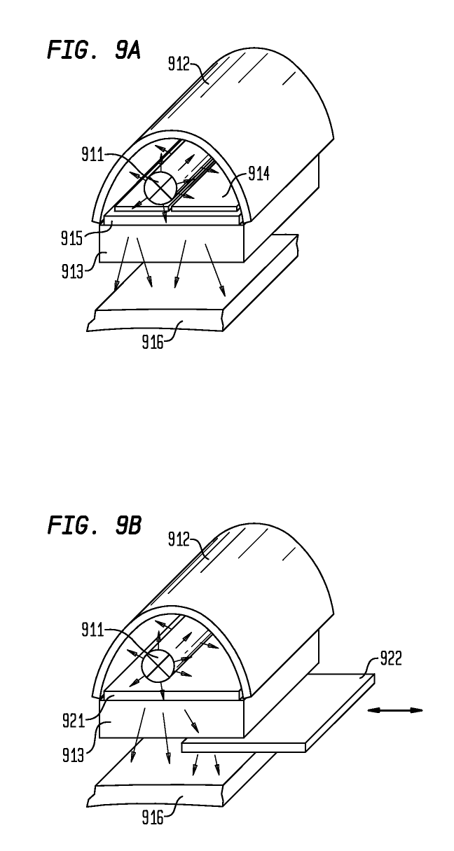

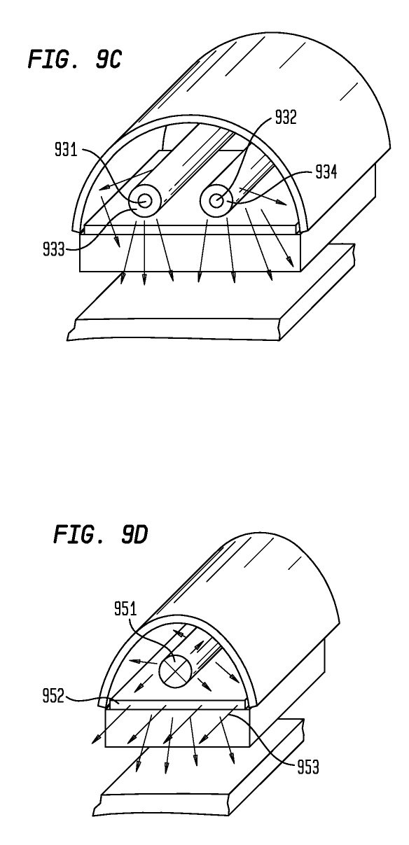

FIGS. 9A-9D illustrate exemplary techniques for obtaining desired combinations of PC, PTV and PTIR pulses from a single applicator (e.g., a handpiece). For example, FIG. 9A schematically illustrates an applicator for implementing the methods of the invention that employs a broadband source 911. A reflector 912 directs the light generated by the light source 911 through a waveguide 913 onto a treatment area 916. A pair of different filters 914 and 915 are placed side-by-side such that each captures a portion of the light reflector by the reflector 912, thereby generating simultaneously output pulses having substantially different spectra. Both pulses can be utilized simultaneously to irradiate a target skin portion.

FIG. 9B illustrates another embodiment of the applicator in which filters 921 and 922 are placed sequentially in the path of the light generated by the light source 911 to produce temporally separated pulses, each having a spectrum dictated by the filtering characteristics of one filter. Hence, varying spectral content of generated pulses can be achieved by switching the filters and/or moving the filters in and out of the light path.

FIG. 9c illustrates another applicator suitable for use in the practice of the invention in which two (possibly different) broadband radiation sources 931 and 932 are employed in combination with two substantially different filters 933 and 934 to generate pulses with different spectral contents.

FIG. 9D schematically illustrates yet another embodiment of an applicator for practicing the methods of the invention having a laser medium 952 that is pumped by a broadband source 951 to generate coherent radiation 953 having desired wavelength components. Two opposing facets of the laser medium 952 can be coated with at least partially reflecting material so as to generate a laser cavity. The medium 952 can serve as a spectral filter providing desired filtration of the radiation output of the broadband source 951 (for example, for forming a PC pulse). Additional filters can also be employed, if necessary. Hence, the coherent radiation can serve, for example, as a PTV or PTIR pulse, depending on the nature of the laser medium. Optical systems known in the art can be employed to direct the radiation 953 to the treatment area. Further, non-linear organic or inorganic crystals can be employed for frequency conversion of light generated by the laser medium to expand the range of the spectral output of the device.

### Enhancement of Treatment

While the techniques discussed are currently preferred, an improved version of some of the prior art techniques involving applying a dye to a follicle is also part of the invention. In the prior art techniques, normal follicles are targeted and the dye migrates around the hair, limiting the amount of dye that reaches the epithelium of the infrainfundibulum and the sebaceous gland, the primary targets for damage or destruction in an acne treatment. By contrast, an atrophic follicle, the follicle primarily targeted for acne treatment has no hair above the sebaceous duct and generally has a wider canal for the infundibulum than normal follicles. Vellus follicles, a secondary target in acne treatment, also have little if any hair in the infundibulum. It is therefore possible to introduce more dye into these follicles, and in particular into the infundibulum, and the infrainfundibulum thereof, and into the sebaceous gland and duct without requiring painful epilation, as compared to other types of follicles. Damage or destruction of the infrainfundibulum's epithelial lining may inhibit comedo formation and thus eliminate acne without destroying the sebaceous gland.

To implement the above, it is preferable that sebum be initially removed mechanically from at least the infundibulum of the follicles. This may be done by pressing the skin adjacent the follicles to be treated to squeeze out the sebum, or by removing the sebum in some other way, such as by suction. The sebum can also be removed chemically, for example, by applying a suitable topical composition to spots to be treated which bonds with the sebum, creating a substance which is easily removed/cleaned. These and other techniques for mechanical cleansing are well known in the art.

A substance is then applied to the treatment area which has an absorption spectrum substantially different from that of the body components/skin in the area. While different dyes can be used for filling the open canal, it must be a biocompatible dye with limited toxic effect, for example food dyes or a dye or compositions used for hair dying: Grecian-5, 5-minute Color Gel (Grecian Formula 16, USA, COMBE Inc., Dist.); Feria 21 (L'OREAL, Paris); Feria 23 (L'OREAL, Paris); Excellence Creme 3 (L'OREAL, Paris); Preference 3 (L'OREAL, Paris); Just for men (USA, COMBE Inc., Dist.); Nice'n Easy 3 (Clairol, USA); Hydrience 3 (Clairol, USA); Lasting Color 2 (Clairol, USA); Loving Care, Color Creme (Clairol, USA); KMnO₄i C₆H₄(NH₂)₂2HCl+H₂O₂; Strong tea+FeCl₃; Universal black dye, TU 2389-0-001-27520934-94 (Russia); Gamma, TU 10-04-16-154-89 (Russia); Henna/Basma—natural dye, TU 9158-014-0033-5018-93 (Russia); Indian ink, TU 6-15-458-86 (Russia); Indian ink with casein; TU 6-15-458-86 (Russia); Chromogene black T (Russia); “Contrast”, TU-6-36-0204187-577-0-89 (Russia); Aniline black dye, TU 6-360204187-466-90, Ursol, Effect of Nature or dyes and compositions used for tattoos. It can also be a preparation of small (1-100 nm) metal particles such as Au, Ag, Cu, Pt, titanium based compounds with strong plasmon resonance effect. Alternatively, fullerenes, carbon nano-tubes or metal-coated dielectric particles can be used. Any other biocompatible nano particles exhibiting singlet oxygen or active radical photoproduction can also be used. Magnetic particles or electro conductive particles can also be used, magnetic or electrical fields being usable to delivery these particles into the open canal and pilosebaceous gland. The larger canals of the targeted follicles means that a substance having a higher viscosity than is usable in some prior art systems can be used, thus enhancing retention of the dye in the follicle.

The third step is to irradiate the treatment area with light having one or more wavelengths that are preferentially absorbed by the substance applied during the prior step. The light wavelength is preferably not strongly absorbed by melanin so as to protect the epidermis, and is not too strongly absorbed by water so as to minimize tissue damage outside the follicle. While the wavelength(s) applied will vary with the dye utilized, wavelengths in the range of 600-1250 nm are preferred, with wavelengths in the range of 20 800-1250 nm being more preferred to minimize epidermal damage. The power/energy used should be below the threshold at which the applied substance is decomposed or otherwise it may lose its ability to effectively absorb the applied radiation. The duration of the applied light pulse should be long enough to coagulate or to otherwise thermally destroy the epithelium of the infrainfundibulum and/or other undesired parts of the follicle. This action may also result in shrinkage of the gland. Suitable radiant exposure to get the chromophore/dye to approximately 100° C. without water evaporation is roughly 10-200 J/cm², with a pulse duration of 1 ms-5 sec., preferably 10 ms-1 sec., and most preferably 100 ms-0.5 sec.

High power oscillating magnetic or electrical fields (radio frequency or microwave) or light can also be used for heating the magnetic or electrical particles and surrounding infundibulum and pilosebaceous gland. A monopoliar electrode can be used for treatment of the infundibulum and the sebaceous gland. In this embodiment, the pilosebaceous unit can be filled by highly conductive lotion in a preliminary step.

Since, as discussed above, it is desirable for preferred embodiments that pressure and/or cooling be applied to the patient's skin in conjunction with the application of at least some of the light energy pulses applied thereto, the delivery head is preferably a contact head, for example one of a number of suitable contact heads known in the art. A number of suitable cooling techniques known in the art may also be used to cool the patient's skin.

Thus, while the invention has been described above with respect to a number of embodiments, the foregoing and other changes in form and detail may be made therein by ones skilled in the art while still remaining within the spirit and scope of the invention which is to be defined only by the appended claims.

1.-63. (canceled)

64. A dermatological system for treating follicles, comprising:
at least a first radiation generating source for irradiating a portion of skin with at least one pulse of photochemical electromagnetic radiation so as to expose a treatment region of at least one follicle to said radiation, and
at least a second source for generating photothermal radiation to heat at least a portion of the treatment region, the second source providing at least one pulse having a pulse width of between about 1 ms and about 100 s and a photothermal radiation exposure of between about 10 J/cm² and about 500 J/cm² to raise a temperature of at least a portion of the treatment region in a range of about 38° C. to about 47° C.

65. The system of claim 64, further comprising a cooling element for cooling at least a portion of the skin during said irradiation of the treatment region.

66. The system of claim 64, further comprising a contact mechanism capable of coupling to said irradiated skin portion to apply a pressure thereto.

67. The system of claim 66, wherein said applied pressure can be in a range of about 10 to about 100 N/cm².

68. The system of claim 64, wherein at least of one of the sources comprises:
a lamp generating radiation having a broad spectrum, and
one or more filters optically coupled to said lamp for selecting at least one photochemical or photothermal wavelength component from the broad spectrum.

69. The system of claim 64, further comprising:
a housing with a handle and an enclosure,
wherein said at least first and said second sources are within the enclosure, and
wherein the photothermal radiation has one or more wavelengths in a range of about 1000 nm to about 1800 nm.

70. The system of claim 64, wherein the photochemical radiation generating source is any of a LED or an array of LEDs.

71. The system of claim 64, wherein the source for generating photothermal radiation is a halogen lamp.

72. The system of claim 64, wherein said photothermal radiation includes one or more wavelengths in a range of about 900 nm to about 1800 nm.

73. The system of claim 64, wherein said photothermal radiation includes one or more wavelengths in a range of about 470 nm to about 650 nm.

74. The system of claim 73, wherein the second source provides at least one pulse of photothermal radiation having a pulse width of between 1 ms and 100 ms and a photothermal radiation exposure of between 10 J/cm² and 50 J/cm².

75. The system of claim 64, wherein the second source provides an irradiance in a range of about 100 W/cm² to about 50,000 W/cm².

76. The system of claim 64 wherein the second source for generating photothermal radiation provides an irradiance of from about 5 W/cm² to about 10,000 W/cm² in a wavelength range of between 900 nm and 1800 nm.

77. The system of claim 64, wherein the second source is configured to generate at least one pulse of photothermal radiation simultaneous with the at least one pulse of photochemical electromagnetic radiation.

78. The system of claim 64, wherein the second source generates at least one pulse of photothermal radiation at a time interval in a range from about 100 ms to several hours from the at least one pulse of photochemical electromagnetic radiation.

79. The system of claim 64, wherein the at least one pulse of photochemical radiation includes one or more wavelengths in a range of about 380 nm to about 700 nm.

80. The system of claim 64, wherein the at least one pulse of photochemical electromagnetic radiation has a pulse width of between 1 ms and 20 s and a photochemical radiation exposure of between 2 J/cm² and 50 J/cm².

81. The system of claim 64, wherein the at least one pulse of photochemical electromagnetic radiation provides an irradiance in a range of about 1 W/cm² to about 900 W/cm².

82. The system of claim 64, further comprising a broadband radiation source and at least one filter optically coupled to said broadband radiation source for selecting from said broadband radiation a spectral component as the first source and another spectral component as the second source.

83. The system of claim 82, wherein the first source comprises a first filter coupled to the broadband radiation source and the second source comprises a second filter coupled to the broadband radiation source.

84. The system of claim 82, wherein the one or more filters are configured to generate pulses having different spectral characteristics.

85. The system of claim 64, wherein a single radiation apparatus comprises said first radiation generating source and said second source for generating photothermal radiation.
